(19) United States
(12) Patent Application Publication
(10) Pub. No.: US 2017/0202917 A1
(43) Pub. Date: Jul. 20, 2017

(54) PHARMACEUTICAL COMPOSITION FOR PREVENTING OR TREATING SKIN RASH

(71) Applicants: DAEWOONG PHARMACEUTICAL CO., LTD., Seongnam-si, Gyeonggi-do (KR); DONG-A UNIVERSITY RESEARCH FOUNDATION FOR INDUSTRY-ACADEMY COOPERATION, Busan (KR)

(72) Inventors: Sung-Yong OH, Busan (KR); Kyung-Hyun MIN, Yongin-si, Gyeonggi-do (KR)

(73) Assignees: DAEWOONG PHARMACEUTICAL CO., LTD., Seongnam-si, Gyeonggi-do (KR); DONG-A UNIVERSITY RESEARCH FOUNDATION FOR INDUSTRY-ACADEMY COOPERATION, Busan (KR)

(21) Appl. No.: 15/314,282

(22) PCT Filed: May 14, 2015
(86) PCT No.: PCT/KR2015/004826

§ 371 Date: Nov. 28, 2016

(30) Foreign Application Priority Data
May 29, 2014 (KR) 10-2014-0064824

(51) Int. Cl. A61K 38/18; A61K 9/06; A61K 9/00

(52) U.S. Cl. CPC A61K 38/1808; A61K 9/0014; A61K 9/06

(57) ABSTRACT

Provided is a method for preventing or treating skin rash comprising administering to a subject in need thereof epidermal growth factor as an active ingredient. The skin rash includes a skin disorder caused by administering an epidermal growth factor receptor inhibitor, such as erlotinib, as an adverse event.

### TECHNICAL FIELD

The present invention relates to a pharmaceutical composition for preventing or treating skin rash. More specifically, the present invention relates to a pharmaceutical composition for preventing or treating skin rash, comprising epidermal growth factor as an active ingredient.

### BACKGROUND ART

The epidermal growth factor receptor (EGFR) is commonly expressed in high levels in a variety of solid tumors, and it is a known regulator of cell proliferation, survival, metastasis, and angiogenesis (Ciardiello F et al., Eur J Cancer 39:1348-1354). The main pharmacological strategies in clinical development for therapeutic inhibition of EGFR include the uses of monoclonal antibodies which inhibit the ligand-receptor binding, and small-molecules which inhibit the activation of the tyrosine kinase domain. Cetuximab (an anti-EGFR monoclonal antibody) and erlotinib (an EGFR tyrosine kinase inhibitor), are the principal inhibitors of EGFR. The key indications for clinical use of cetuximab are colorectal cancer and head and neck cancer, and erlotinib is the drug of choice for non-small cell lung cancer (NSCLC) and pancreatic cancer (PC) (Shepherd F A et al., N Engl J Med 353:123-132; Bonner J A et al., N Engl J Med 354:567-578; Cunningham D et al., N Engl J Med 351:337-345; Zhou C et al., Lancet Oncol 12:735-742; and Moore M J et al., J Clin Oncol 25:1960-1966). Recent clinical trials have provided substantial evidence that erlotinib, gefitinib, and afatinib should be the standard of care in treating patients with EGFR mutation-positive NSCLC, and that these agents should be considered as first-line treatment options (Zhou C et al., Lancet Oncol 12:735-742; Maemondo M et al., N Engl J Med 362:2380-2388; and Sequist L V et al., J Clin Oncol 31:3327-3334). An analysis of the quality of life (QoL) and time to deterioration based on patient-reported symptoms showed clinically meaningful benefits which were statistically significant, in patients randomized to erlotinib treatment (Bezjak A et al., J Clin Oncol 24:3831-3837).

Skin rash is a common side-effect of all EGFR inhibitors (Lynch T J, Jr. et al, Oncologist 12:610-621; and Li T et al., Target Oncol 4:107-119). The development of a rash above the waist is the most common adverse event associated with erlotinib, and the rash generally develops within 7-10 days of starting treatment (Lynch T J, Jr. et al., Oncologist 12:610-621). The skin rash may spontaneously resolve without treatment, and reappears following continuation of the treatment. This chronic side effect is very distressing for the patient (Joshi S S et al., Cancer 116:3916-3923). Xerosis is also commonly observed in patients on EGFR inhibitor therapy. Patients develop a scaly, dry, pruritic skin in varying degrees on any part of the body, including ocular, perineal, and vaginal areas. Itching as a result of dry skin and pruritis can often lead to superinfection, resulting in cellulitis and folliculitis. Xerosis can also lead to swelling and cracking of lips, mucosal irritation, erythema, and inflammation (Galimont-Collen A F et al., Eur J Cancer 43:845-851). EGFR inhibitor therapy is also associated with paronychia which can occur in about 6-12% of patients, most commonly affecting the nail bed of the great toe (Lynch T J, Jr. et al., Oncologist 12:610-621). These nail changes lead to inflammation, tenderness, formation of pyogenic granuloma-type lesions, and fissuring of lateral nail folds or distal finger nail bed.

The overall incidence of erlotinib-related skin effects (ERSEs) was 75% in NSCLC and PC clinical trials (grade 3-4, in about 10% of the patients) (Lynch T J, Jr. et al., Oncologist 12:610-621; Li T et al., Target Oncol 4:107-119; Thatcher Net al., Oncologist 14:840-847; and Gridelli C et al., Crit Rev Oncol Hematol 66:155-162). In most of the patients, ERSEs could affect the quality of life, which often results in treatment dose adjustments or temporary interruptions of treatment (Joshi S S et al., Cancer 116:3916-3923; and Lacouture M E et al., Support Care Cancer 18:509-522). Even though dermatological reactions could be surrogate markers for survival prediction (Perez-Soler R et al., J Clin Oncol 22:3238-3247), they cause significant physical and psycho-social discomfort to patients (Joshi S S et al., Cancer 116:3916-3923). Etiological investigations of rash management should be a high priority, given the increasing use of EGFR-targeted agents, particularly erlotinib, in the treatment of NSCLC and PC.

Hence, there is a need to develop a method capable of inhibiting the skin rash derived from the use of EGFR-targeted agents, especially erlotinib, in NSCLC and PC treatment.

### DISCLOSURE

### Technical Problem

The present inventors carried out clinical trials on ERSE problems, especially skin rash, using epidermal growth factor (EGF). As a result thereof, it has been found that epidermal growth factor can inhibit the skin rash caused by erlotinib treatment as a side effect, in a statistically significant manner.

Therefore, the present invention provides a pharmaceutical composition for preventing or treating skin rash, comprising epidermal growth factor.

### TECHNICAL SOLUTION

In accordance with an aspect of the present invention, there is provided a pharmaceutical composition for preventing or treating skin rash, comprising epidermal growth factor as an active ingredient.

In the pharmaceutical composition according to the present invention, the skin rash may be a skin disorder caused by administering an epidermal growth factor receptor inhibitor, e.g., erlotinib. And also, the pharmaceutical composition according to the present invention may be in a dosage form for topical administration, e.g., in an ointment.

### ADVANTAGEOUS EFFECTS

It has been found by the present invention that skin rashes, especially the skin rash caused by administering an epidermal growth factor receptor inhibitor such as erlotinib as an adverse event, can be effectively prevented and/or treated by topical administration of epidermal growth factor. Therefore, the pharmaceutical composition according to the present invention can be usefully applied for preventing or treating skin rash.

### DESCRIPTION OF DRAWINGS

FIG. 1 shows the 74-year-old female patient with non-small cell lung cancer treated with erlotinib (150 mg). In the FIG. 1(a), erythematous patch with pustules and crust were observed on seborrheic and perioral area. FIG. 1(b) shows improved skin lesions following 4 weeks treatment.

FIG. 2 shows histopathological findings of FIG. 1. FIG. 2(a) shows moderate to severe dense perivascular lymphohistiocytic cell infiltration, where perifollicular inflammation with dilated vessels with extravasated red blood cells at baseline was noted. FIG. 2(b) shows markedly reduced inflammatory cell infiltration after 4 weeks of treatment.

FIG. 3 shows the 44-year-old male with pancreatic cancer treated with erlotinib (100 mg) and gemcitabine. In the FIG. 3(a), disseminated papules and pustules were observed on the face. In the FIG. 3(b), only mild erythema and small papules were observed four weeks after the treatment with the EGF ointment.

### BEST MODE

The present invention provides a pharmaceutical composition for preventing or treating skin rash, comprising epidermal growth factor as an active ingredient.

In the pharmaceutical composition according to the present invention, the term “epidermal growth factor (EGF)” refers to a protein known to facilitate epidermis regeneration, including any and all native proteins and/or recombinant proteins (e.g., rh-EGF) known in the art.

The term “skin rash” refers to a change of the skin which affects its color, appearance, or texture. The skin rash may be localized on one part of the body, or affect all the skin. The skin rash may be also referred to as ‘hives’. The skin rash results from various causes, including allergy, dermatitis, etc. In the present invention, the term “skin rash” includes skin rashes resulting from any and all causes.

In an embodiment, the skin rash may be a skin disorder caused by administering an epidermal growth factor receptor inhibitor as an adverse event. The epidermal growth factor receptor inhibitor includes an EGFR-tyrosine kinase inhibitor such as erlotinib, gefitinib, lapatinib, etc., but not limited thereto. Preferably, the pharmaceutical composition according to present invention may be usefully applied for the skin rash caused by administering erlotinib as an adverse event.

The pharmaceutical composition of the present invention may be formulated into various dosage forms, along with pharmaceutically acceptable excipients. The pharmaceutical composition of the present invention may be formulated preferably into a dosage form for topical administration, such as solution for external use, emulsion, ointment, cream, lotion, patch, etc., more preferably into ointment. The pharmaceutical composition of the present invention can be administered to patients who suffer from various skin rashes at a daily dosage of about 0.1 to 100 ppm/kg. An adequate dosage is generally changed according to age, body weight, and conditions of a patient.

The present invention will be described in further detail with reference to the following examples. These examples are for illustrative purposes only and are not intended to limit the scope of the present invention.

1. Patients and Methods

(1) Patients

The current study included patients diagnosed with advanced NSCLC or PC, having histopathological confirmation. The inclusion criteria were NSCLC treated with erlotinib alone and PC treated with gemcitabine and erlotinib combination chemotherapy, and patients should have sufficient liver, kidney, and bone marrow functions to undergo treatment. All the patients had Grade ERSEs according to the National Cancer Institute's Common Terminology Criteria for Adverse Events (NCI-CTCAE) v. 3.0. The study was approved by the Institutional Review Board of all the participating centers, and the patients were provided written informed consent before enrolment.

(2) Treatment and Evaluation

EGF ointment (Saesal Yongo™, Daewoong Pharmaceuticals Co. Ltd.) containing 1 ppm of nepidermin, was evenly applied to the skin lesions twice a day for patients with Grade ≧2 lesions. Skin toxicity was categorized by rash/desquamation, rash/acne, dry skin, itching, or nail change. The patients visited an outpatient clinic according to a planned chemotherapy schedule every three or four weeks. The efficacy of the treatment was not assessed until at least one week after the application of the ointment. The effectiveness of the EGF ointment was defined as follows: (1) Grade 2, 3, or 4 ERSEs downgraded to ≦Grade 1 or (2) Grade 3 or 4 ERSEs downgraded to Grade 2 and sustained for at least two weeks. If the skin lesions showed no improvement after application of the EGF ointment for eight weeks, the treatment was stopped and classified as “no effect”. If the patients required medication to control infection and itching, administration of oral and I.V. antibiotics, antihistamine drugs, and steroids were allowed during this study. However, topical steroids or topical antibiotics were not permitted. Dermatological toxicity was assessed according to the NCI-CTCAE v.3.0. The QoL was evaluated with SKINDEX-16. The use of SKINDEX-16 was approved by the Mapi Research Trust.

(3) Statistical analysis

This was an open label, non-comparative, multicenter, phase II trial. A previous study had reported an improvement of 30% in skin rashes following prophylactic treatment with tetracycline, when compared with placebo (Jatoi A et al., Cancer 113:847-853). This trial was designed to identify whether the effectiveness of EGF ointment is 20% more than the prior treatment. p0=0.3, p1=0.5, a two-stage optimal design was adopted, as previously proposed by Simon (Simon R, Control Clin Trials 10:1-10) with a statistical power of 80% for hypothesis acceptance and 5% significance for hypothesis rejection. The number of patients needed for the first stage was calculated as 15. If six or more ERSEs occurred, the study was to be terminated. Assuming a 10% follow-up loss, the total sample size required was 52 patients. Categorical variables in the two groups were compared via x² tests or Fisher's exact tests. P values less than 0.05 were regarded as statistically significant, and all P values corresponded to two-sided significance tests. All data were analyzed with IBM SPSS software v. 20.0.

2. Results

(1) Patients' characteristics

Between October 2012 and November 2013, 52 patients from seven institutes in Korea were enrolled, after obtaining informed consent. Among these patients, 6 patients dropped out after 14 days, before the effectiveness of the EGF ointment could be evaluated: 3 were lost to follow-up, 1 wished to withdraw informed consent, and 2 patients did not state the reason for leaving the study. Of the 46 patients evaluated, the male: female ratio was 30:16. The median age was 61 years (range: 40-83 years); 31 (67%) patients had NSCLC, and 15 (33%) patients had PC. Most of the patients had good performance status (PS) (based on the Eastern Cooperative Oncology Group (ECOG) criteria): PS 0-1: (93%). No history of prior chemotherapy was reported in 20 (43%) patients, and 26 (57%) patients had received one or more sessions of palliative chemotherapy (Table 1).

TABLE 1
Patients′ characteristics

| | | N = 46 | % |
|---|---|---|---|
| Gender | Male | 30 | 65 |
| | Female | 16 | 35 |

| | | |
|---|---|---|
| Age | Median (range) | 61 (10-83) |

| | | | |
|---|---|---|---|
| | ≦60 | 20 | 43 |
| | >60 | 26 | 57 |
| PS (ECOG) | 0-1 | 43 | 93 |
| | 2 | 3 | 7 |
| Cancer type | NSCLC | 31 | 67 |
| | PC | 15 | 33 |
| Previous number of | 0 | 20 | 43 |
| chemotherapy sessions | 1 | 17 | 37 |
| | >2 | 9 | 20 |

(2) Assessment of the effect of the EGF ointment

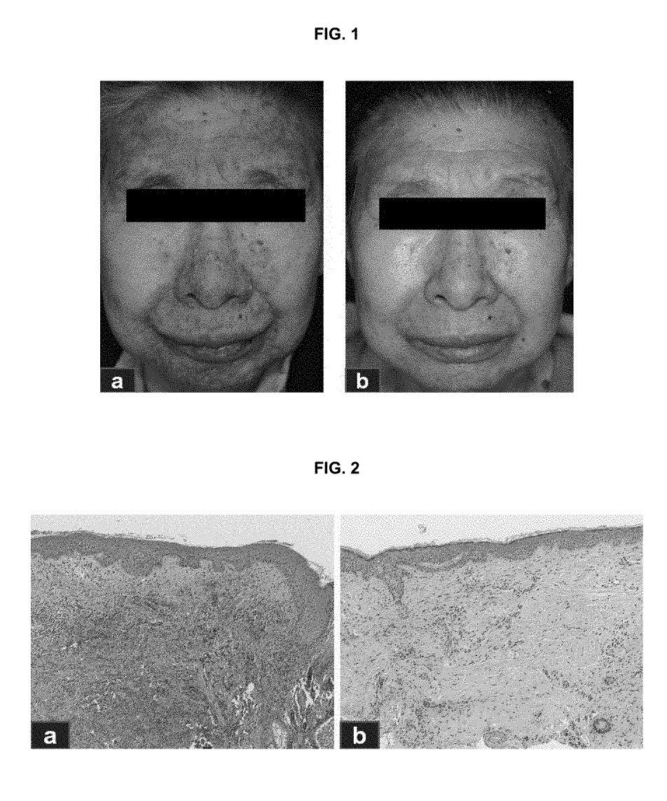

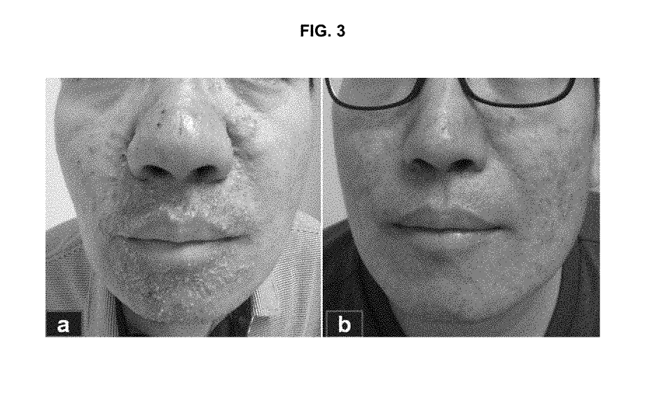

The final evaluation included 46 patients (30 males, 16 females). According to the definition used, the EGF ointment was effective in 36 (69.2%) intention to treat patients for the treatment of ERSEs (FIGS. 1-3). Among 36 responded patients, 31 patients were by first criteria of effectiveness. 5 patients were by criteria 2. The other 10 patients showed no effect with the EGF ointment. Rash/acne and itching were common causes for enrolling in the study (Table 2).

TABLE 2
Patients′ skin status at study enroll

| | NCI-CTCAE Grade* | | | | |
|---|---|---|---|---|---|
| | 0 | 1 | 2 | 3 | 4 |
| Rash/desquamation | 19 | 4 | 18 | 4 | 1 |
| Rash/acne | 4 | 2 | 30 | 9 | 1 |
| Dry skin | 19 | 6 | 21 | | |
| Itching | 7 | 11 | 25 | 3 | |
| Nail change | 36 | 6 | 4 | | |

*NCI-CTCAE: National Cancer Institute's Common Terminology Criteria for Adverse Events version 3.0

The grading for the average NCI-CTCAE rating of rash/acne and itching improved from 2.02±0.83 to 1.13±0.89, and from 1.52±0.84 to 0.67±0.90, respectively (p<0.001) (Table 3).

TABLE 3
Change in NCI-CTCAE v. 3.0 grade

| | Initial | Max. response | | |
|---|---|---|---|---|
| | Mean ± SD | Mean ± SD | Improvement | |
| | (95% CI) | (95% CI) | (%) | p value* |
| Rash/desqua- | 1.22 ± 1.15 | 0.54 ± 0.81 | 55.7 | <0.001 |
| mation | (0.87 to 1.56) | (0.30 to 0.78) | | |
| Rash/acne | 2.02 ± 0.83 | 1.13 ± 0.89 | 44.1 | <0.001 |
| | (1.78 to 2.27) | (0.87 to 1.39) | | |
| Dry skin | 1.04 ± 0.94 | 0.63 ± 0.80 | 39.4 | 0.001 |
| | (0.76 to 1.32) | (0.39 to 0.87) | | |
| Itching | 1.52 ± 0.84 | 0.67 ± 0.90 | 55.9 | <0.001 |
| | (1.27 to 1.77) | (0.41 to 0.94) | | |
| Nail change | 0.30 ± 0.62 | 0.15 ± 0.47 | 50.0 | 0.018 |
| | (0.12 to 0.49) | (0.01 to 0.29) | | |

*P-value was calculated by paired t-test for difference before and after treatment

No statistically significant differences were observed in the effectiveness of the EGF ointment application, based on gender, age, tumor type, erlotinib dose and number of prior chemotherapy sessions (p=NS). The skin rashes in the PC (p=0.085) patients or in those treated with 100 mg of erlotinib (p=0.117) seemed to show a better response; however, these findings were not statistically significant (Table 4).

TABLE 4
Factors analysis of effectiveness of EGF ointment

| Factor | No effect | Effect | p value* |
|---|---|---|---|
| Gender (male/female) | 6/4 | 24/12 | 0.485 |
| Age (>60/≦60) | 6/4 | 20/16 | 0.547 |
| Cancer type (NSCLC/PC) | 9/1 | 22/14 | 0.085 |
| Dosage of erlotinib (150 mg/100 mg) | 8/2 | 19/17 | 0.117 |
| Prior number of chemotherapy (0/≧1) | 4/6 | 16/20 | 0.547 |
| Co-medication (−/+) | 8/2 | 25/11 | 0.700 |

*Fisher's exact tests

The most common reason for discontinuing the study was progression of cancer (37%); 11 patients stopped applying the EGF ointment due to no effect or other side-effects. Of these, 2 patients declined the treatment due to adverse side-effects, 1 patient complained of an uncomfortable feeling of skin stretching, and 1 patient had a feeling of periungual skin overgrowth (Table 5). The clinician prescribed co-medication to 13 patients: 6 patients received antihistamine (ucerax, azeptin), 4 patients were given oral antibiotics (minocycline), and 3 patients were prescribed both. However, there was no difference in the effectiveness of the EGF treatment in the patients who received the co-medication and those who did not.

TABLE 5
Reasons of discontinuation

| Reasons | | Number | % |
|---|---|---|---|
| EGF ointment factors | No effectiveness | 9 | 19.6 |
| | EGF ointment side effect | 2 | 4.3 |
| Non-EGF ointment factors | Disease progression | 17 | 37.0 |
| | Follow up loss or death | 6 | 13.0 |
| | Chemotherapy stopped | 1 | 2.2 |
| | Patient's violation | 1 | 2.2 |
| Others | Resolved ERSEs | 7 | 15.2 |
| | On going* | 3 | 6.5 |

*On going: applying EGF ointment without discontinuation

(3) Quality of Life (QoL) evaluation with Skindex-16

The QoL of 25 patients from two institutions (Dong-A University Hospital and Samsung Medical Centre) was assessed. The Skindex-16 score was evaluated at the beginning of the study and for every visit during cancer treatment. The results of the Skindex-16 were analyzed as an overall score and three domain scores (including symptoms, emotions and functioning), and reported as medians and semi-interquartile ranges (SIQR) (half the distance between the 25th and 75th percentiles). The study population had a median overall Skindex-16 score of 41.25 (SIQR, 14.38). The highest scores were for the emotions domain (median score, 42.86; SIQR, 15.71), which was higher than the functioning domain (median score, 40.0; SIQR, 20.0) and symptoms domain (median score, 35.0; SIQR, 20.0). The median overall Skindex-16 score after the treatment was 8.75, which was significantly lower than the initial overall median Skindex-16 score of 41.25 (p=0.0019). The median Skindex-16 scores in the domain response also decreased as compared to the baseline scores; the levels of symptoms, emotions, and functioning domains decreased to 15.00 (p=0.0137), 11.43 (p=0.0023), and 4.00 (p=0.0026), respectively (Table 6).

TABLE 6
Summary of SKINDEX-16 Domain Analysis

| | | | | | Total |
|---|---|---|---|---|---|
| Variables | Statistic | Symptoms | Emotions | Functioning | (N = 25) |
| baseline | Mean ± SD | 40.20 ± 23.65 | 43.66 ± 21.70 | 40.16 ± 25.75 | 41.70 ± 20.01 |
| | Median | 35.00 | 42.86 | 40.00 | 41.25 |
| | SIQR(Range) | 20.00(5.00 to 90.00) | 15.71(8.57 to 85.71) | 20.00(0.00 to 100.00) | 14.38(12.50 to 91.25) |
| | P-value* | 0.6847 | | | |
| Maximum | Mean ± SD | 22.60 ± 23.90 | 23.31 ± 29.78 | 17.12 ± 29.27 | 21.20 ± 27.06 |
| response | Median | 15.00 | 11.43 | 4.00 | 8.75 |
| (after treatment) | SIQR(Range) | 15.00(0.00 to 80.00) | 14.29(0.00 to 100.00) | 8.00(0.00 to 100.00) | 10.63(0.00 to 91.25) |
| | P-value* | 0.0833 | | | |
| Change | Mean ± SD | −17.60 ± 33.08 | −20.34 ± 29.82 | −23.04 ± 34.32 | −20.50 ± 29.39 |
| from | Median | −10.00 | −17.14 | −20.00 | −20.00 |
| baseline | SIQR(Range) | 20.00(−90.0 to 40.00) | 12.86(−85.71 to 51.43) | 24.00(−100.0 to 56.00) | 11.88(−91.25 to 50.00) |
| | P-value† | 0.0137 | 0.0023 | 0.0026 | 0.0019 |
| | P-value* | 0.5310 | | | |

*P-value from repeated measure ANOVA between symptoms, emotions, and functions.
†P-value was calculated by paired t-test for difference before and after treatment.

3. Conclusion

From the above results, it can be seen that the EGF ointment is effective to ERSEs regardless of gender, age, type of tumor, and dosage of erlotinib. And also, the EGF ointment evenly improves all kind of symptoms of ERSE.

1.-5. (canceled)

6. A method for treating skin rash in a subject in need thereof comprising administering an effective amount of epidermal growth factor to the subject.

7. The method of claim 6, wherein the skin rash is caused by administering an epidermal growth factor receptor inhibitor.

8. The method of claim 7, wherein the epidermal growth factor receptor inhibitor is erlotinib.

9. The method of claim 6, wherein the epidermal growth factor is topically administered.

10. The method of claim 9, where the epidermal growth factor is administration in a dosage form of ointment.
